(19) United States
(12) Patent Application Publication
(10) Pub. No.: US 2025/0298265 A1
(43) Pub. Date: Sep. 25, 2025

(54) METHOD FOR COMPENSATING THE TRAVEL TIME DIFFERENCES OF IMAGE WAVEGUIDES

(71) Applicant: TECHNISCHE UNIVERSITÄT DRESDEN, Dresden (DE)

(72) Inventors: Robert KUSCHMIERZ, Dresden (DE); Jürgen CZARSKE, Dresden (DE); Elias SCHARF, Dresden (DE); Martin KROLL, Dresden (DE)

(73) Assignee: TECHNISCHE UNIVERSITÄT DRESDEN, Dresden (DE)

(21) Appl. No.: 19/058,932

(22) Filed: Feb. 20, 2025

(30) Foreign Application Priority Data
Mar. 20, 2024 (EP) 24164836

(51) Int. Cl. G02F 1/01

(52) U.S. Cl. CPC G02F 1/0126

(57) ABSTRACT

The invention relates to a method and a device for compensating for the travel time differences of image waveguides and/or for implementing a desired travel time profile, as well as to the use of the method and the device. The method involves changing the effective refractive indices of optical fibers by means of high-energy electromagnetic radiation, which are enclosed by an image waveguide. Possible applications of the method and device include, but are not limited to, cancer diagnostics, nonlinear endomicroscopy, optical coherence tomography (OCT), optical coherence tomography with tuned wavelength of the radiation source (swept source OCT), the undisturbed transmission of femtosecond pulses and/or the correction of travel time differences that occur in image waveguides that have optical fibers twisted with each other.

### CROSS-REFERENCE TO RELATED APPLICATIONS

This application claims priority to European application nₒ. EP 24 164 836, which was filed on 20 Mar. 2024, the entire contents of which are hereby incorporated by reference herein.

The invention relates to a method and a device for compensating for the travel time differences of image waveguides and/or for implementing a desired travel time profile, as well as to the use of the method and the device. The method involves changing the effective refractive indices of optical fibers by means of high-energy electromagnetic radiation, which are enclosed by an image waveguide. Possible applications of the method and device include, but are not limited to, cancer diagnostics, nonlinear endomicroscopy, optical coherence tomography (OCT), optical coherence tomography with tuned wavelength of the radiation source (swept source OCT), the undisturbed transmission of femtosecond pulses and/or the correction of travel time differences that occur in image waveguides that have optical fibers twisted with each other.

### BACKGROUND OF THE INVENTION

Endoscopes for imaging and illumination are used in medical technology for minimally invasive diagnostics in difficult-to-access areas, which is why it is advisable to keep their diameter as small as possible (the target size is less than 0.5 mm) and their mechanical flexibility as high as possible. They also require a high contrast, a high spatial resolution and reliability, as well as suitable optical imaging modalities and low costs. Especially in nonlinear imaging methods such as nonlinear endomicroscopy, light pulses with high pulse power density are required, which requires image waveguides with a travel time profile that is as short as possible in the time domain. “Travel time profile” refers here to the set of all travel time differences between the travel times of the optical fibers of an image waveguide and a reference travel time. The reference travel time here can be the mean or median of all travel times of a plurality of optical fibers of the image waveguide, the travel time of any fiber, or the travel time of an external signal. Other reference travel times are not excluded.

Prior art borescope endoscopes are known which are based on rod and gradient index lenses (GRIN lenses-lenses in which the refractive index changes as a function of the distance from the center of the lens) and provide two-dimensional images of the intensity of electromagnetic radiation from the distal end (the application side) to the proximal end (the instrument side). Such endoscopes have, for functional reasons, rigid optical waveguide arrangements with diameters of more than 1 mm. This precludes applications in neurosurgery, for example.

In addition, the prior art includes camera endoscopes. These offer a high degree of flexibility because the camera and an illumination unit are located at the distal end, and only electrical signals need to be transmitted to the proximal end. The minimum endoscope diameter here is 2 mm. Camera endoscopes likewise allow two-dimensional imaging, and no flexible lighting. Three-dimensional imaging is made possible by stereo camera systems, but requires a larger endoscope diameter of about 10 mm. Furthermore, the electromagnetic compatibility of camera endoscopes may be inadequate.

In nonlinear endomicroscopy, single-mode optical waveguides are usually used. Single-mode optical waveguides have only one spatial transmission channel, and for this reason require complex 2D/3D scanning optics at the distal end. This limits the minimum diameter to several millimeters. The scanning optics have a limited range of applications in terms of image field diameter and wavelength, and are associated with high costs. Conventional endoscopes have coherent bundles of optical waveguides—also known as coherent fiber bundles (CFB)—which contain about 10,000 to 100,000 fiber cores. An ordered fiber bundle is referred to as “coherent” if the positional relationship between any two fibers of the bundle is maintained over the entire length of the bundle. Such endoscopes allow an undistorted transmission of the two-dimensional intensity distribution in the plane of the distal fiber end surface. Planes of the inspection region can be imaged by integrating rigid, macroscopic imaging optics on the distal fiber end surface. The relative spatial resolution is determined by the number of fiber cores. Distal imaging optics can increase the absolute spatial resolution, but reduce the image field diameter. The minimum endoscope diameter is limited to the millimeter range by the necessary distal imaging optics.

CFB endoscopes without complex imaging optics in the distal measuring head would make possible an endoscope diameter of less than 500 μm, because it would only be limited by the fiber diameter. When a planar wave of electromagnetic radiation strikes one end of a CFB, the radiation can have a different travel time and a different phase upon exiting each fiber at the other end of the CFB. This is because of the scattering of the material parameters, such as the effective refractive index of the individual fibers. Effective refractive indices are usually wavelength-dependent. The difference in the travel time between the radiation exiting one fiber at the other end of the CFB and a reference travel time or the travel time of the exiting radiation averaged over all fibers is called travel time difference. The set of travel time differences of all fibers of the CFB is called the travel time profile of the CFB. It is also possible to define travel time profiles of subsets of all fibers of the CFB. The scattering of the travel time of the fibers of a CFB prevents the undisturbed transmission of femtosecond pulses and expands them in the time domain. Since fiber travel times are proportional to the optical path length, the lengths of CFB for use in 2-photon microscopy or 2-photon ablation are limited to about 10 cm. In medicine, however, fiber optic endoscopes with lengths of several meters are often required, for example in brain examination procedures that are based on magnetic resonance imaging.

The phase difference between the radiation exiting one fiber at the other end of the CFB and the phase of the exiting radiation averaged over all fibers is called phase distortion. The set of phase distortions of all fibers of the CFB is called the phase distortion profile. Each CFB can have a different travel time profile and a different phase distortion profile, which is why the temporal resolution of a signal is reduced and the phase information of the electromagnetic radiation is lost. Only two-dimensional images with a fixed image plane are therefore possible. For high-resolution three-dimensional imaging, the most commonly investigated approach is to measure the travel time differences phase distortions of a CFB and to compensate for them using a digital optical phase conjugation by means of programmable, digital, optical spatial light modulators (SLM). Spatial light modulators are adaptive elements that allow the phase modulation of electromagnetic radiation. For example, they may comprise arrangements of micromirrors that can be separately controlled, lowered raised and/or tilted. Spatial light modulators can also be embodied as liquid crystals on a silicon substrate—also known as liquid crystal on silicon (LCoS). By applying a voltage to individual crystals of an LCOS, their refractive index can be changed. LCOS can be designed to transmit and/or reflect electromagnetic radiation. The disadvantages of spatial light modulators are that they have low photon efficiency and robustness, as well as being expensive and involved to adjust.

One method in which the change of the effective refractive indices of optical fibers is implemented by means of high-energy electromagnetic radiation is the production of so-called fiber Bragg gratings. These are spots that occur periodically along the length of an optical fiber whose effective refractive index is different from that of the rest of the fiber. Light coupled into an optical fiber having a fiber Bragg grating, whose wavelength equals approximately twice the grating period multiplied by the effective refractive index, is partially reflected at each grating element. The manufacturing method of such a grating involves illuminating portions of the fiber longitudinally at regular intervals with UV light, which is capable of changing the effective refractive index of the fiber material. Due to the required longitudinal illumination of fibers, the manufacturing method is not suitable for changing the optical properties of an optical waveguide after its production. In addition, fiber Bragg gratings are suitable for filtering individual wavelengths, but not for compensating for differences in travel time or for any other precise, targeted change in the travel time profile of an optical waveguide.

From the publication by Yoshinari Maezono et al., it is known that the refractive index of germanium-doped silicon dioxide fibers exhibits a higher photosensitivity to UV radiation of the wavelengths 172 nm and 146 nm when they have been previously loaded with hydrogen. The radiation was used to create fiber Bragg gratings from Xe₂* or Kr₂* excimer lamps. A disadvantage of the method is that it is suitable for filtering out radiation of individual wavelengths more effectively during transmission within optical fibers, but not for specifically controlling the travel time of the radiation. Likewise, the fiber Bragg gratings cannot be subsequently created or modified after an optical waveguide, including the outer sheath, has been manufactured.

In Lancry et al., the relationship between the chemical composition of optical fiber preforms and threshold values of femtosecond laser pulse energies is described, which cause changes in the refractive indices of the preforms that consist of doped silicon dioxide glasses. Disadvantageously, the publication does not reveal how the effect can be used for image waveguides to specifically change their travel times or travel time profiles.

The publication US 2021/0382290 A1 discloses a device for transporting and controlling light beams that comprises an optical waveguide that has a bundle of single-mode optical fibers, wherein each single-mode optical fiber is designed to receive a light beam at a proximal end and to emit a light beam at a distal end, the bundle of single-mode optical fibers having, during operation, a minimum radius of curvature that corresponds to a maximum curvature of the fiber bundle. The device further comprises a phase control SLM that is arranged on the side of the proximal end of the optical waveguide and is suitable for applying a phase shift to each of the light beams to be received at the proximal end in order to form an illumination beam with a predetermined phase function at the distal end of the optical waveguide. The bundle of single-mode optical fibers is twisted and has a twist period that is suitable to maintain the phase function and the travel time profile of the bundle at the distal end of the optical waveguide when the bundle of single-mode optical fibers is subjected to a curvature that is less than the maximum curvature. The device is suitable for guiding optical pulses with a pulse duration between 100 fs and 10 ns. A disadvantage is that, due to the twisting, the optical fibers of the bundle have additional travel time differences, even if these remain constant while bending the bundle.

The publication US 2022/0248938 A1 discloses an optical system and an imaging method. The optical system comprises a multifiber waveguide consisting of multiple optical waveguides and an optical diffuser that allows an intensity pattern to be projected onto the multifiber waveguide. The intensity pattern represents phase information of light emitted by at least one three-dimensional object. The waveguide is designed to transmit the intensity pattern in the form of a large number of pixels to an evaluation system. The evaluation system is designed to generate an image of the object, wherein the generation is based on the intensity pattern transmitted via the waveguide. The disadvantage is that the complex-valued transfer function of the system cannot be defined and the 3D imaging must be obtained exclusively from intensity information.

The publication by Mirsky & Shaked presents a system for overcoming the limitation of the field of view in off-axis holography. The use of a Mach-Zehnder interferometer for off-axis multiplexing is mentioned. The disadvantage is that the system is not suitable for compensating for the travel time differences of image waveguides.

### BRIEF SUMMARY OF THE INVENTION

The object of the invention is therefore to provide a method and a device which overcome the disadvantages of the prior art by making it possible to shorten the travel time differences between optical fibers of image waveguides or to apply a specific travel time profile to image waveguides.

### BRIEF DESCRIPTION OF THE DRAWINGS

The invention is explained in more detail with reference to the drawings. In the drawings:

FIG. 1 shows a schematic representation of an image waveguide (1) having four optical fibers (2, 2.1), wherein the first and fourth optical fibers have an effective refractive index of nₒ, the second optical fiber has an effective refractive index<nₒ, and the third optical fiber has an effective refractive index>nₒ. Furthermore, FIG. 1 shows an incoming pulse of electromagnetic radiation (6) which is simultaneously coupled into all optical fibers of the image waveguide (1), which, due to different effective refractive indices of the optical fibers, is coupled out from all optical fibers of the image waveguide (1) as a non-simultaneously coupled-out exiting pulse of electromagnetic radiation (7) in such a way that it leaves the second optical fiber first, then leaves the first and fourth optical fibers simultaneously, and leaves the third optical fiber last. The first subset of optical fibers (2.1) corresponds here to all the optical fibers (2) of the image waveguide (1).

FIG. 2 shows a schematic representation of the image waveguide (1) having four optical fibers (2, 2.1, 2.3) from FIG. 1, which is exposed to high-energy electromagnetic radiation (8) in order to achieve a desired travel time profile. The desired travel time profile is characterized in that the first, the second as well as the fourth optical fiber have the same effective refractive index, and the third optical fiber has a higher effective refractive index, whereby a pulse coupled in at the first end of the image waveguide (1) is simultaneously coupled out of the first, the second, as well as the fourth optical fiber at the second end of the image waveguide (1), and is coupled out from the third optical fiber with a delay. Since the third optical fiber already has the highest effective refractive index and the first as well as the fourth optical fibers have the median value of the effective refractive indices, the refractive index of the second optical fiber is matched to that of the first and fourth optical fibers. Therefore, the high-energy electromagnetic radiation (8) of a source (5) is coupled into the second optical fiber (2.2) in order to achieve the desired travel time profile of the third subset of optical fibers (2.3) of the image waveguide (1). The second subset (2.2) comprises the second optical fiber, and the third subset of optical fibers (2.3) in this case comprises all the optical fibers (2) of the image waveguide (1).

FIG. 3 shows a schematic representation of the image waveguide (1) having four optical fibers (2, 2.1, 2.3) from FIG. 2 after the high-energy electromagnetic radiation (8) has been coupled into the second optical fiber (2.2), and a simultaneously coupled-in incoming pulse of electromagnetic radiation (6). Due to the previous coupling in of high-energy electromagnetic radiation (8), the effective refractive index of the second optical fiber (2.2) has reached the value nₒ, and the exiting pulse of electromagnetic radiation (7) has the desired travel time profile.

FIG. 4 shows a schematic representation of the image waveguide (1) having four optical fibers (2, 2.1, 2.3) from FIG. 1, which is exposed to high-energy electromagnetic radiation (8.0, 8.1) in order to compensate for the travel time differences of the third subset of optical fibers (2.3) of the image waveguide (1). Since the third optical fiber already has the highest effective refractive index and the first, second, and fourth optical fibers have lower effective refractive indices, the refractive index of the first, second, and fourth optical fibers is matched to that of the third optical fiber. Therefore, the high-energy electromagnetic radiation (8.0, 8.1) of a source (5) is coupled into the second subset (2.2) comprising the first, the second, and the fourth optical fiber (2.2). Since the effective refractive index of the second optical fiber is lower than that of the first and fourth optical fibers, the electromagnetic radiation (8.1) that is coupled into the second optical fiber has a higher energy than the electromagnetic radiation (8.0) coupled into the first and third optical fibers, which has a lower energy, in order to achieve compensation of the travel time differences of the image waveguide (1). The coupling in of the high-energy electromagnetic radiation (8.0, 8.1) takes place in any temporal order. The third subset of optical fibers (2.3) here includes all optical fibers (2) of the image waveguide (1).

FIG. 5 shows a schematic representation of the image waveguide (1) having four optical fibers (2, 2.1, 2.3) from FIG. 4 after the high-energy electromagnetic radiation (8.0) having less energy has been coupled into the first and fourth optical fibers (2.2), as well as the high-energy electromagnetic radiation (8.1) having more energy has been coupled into the second optical fiber (2.2), and a simultaneously coupled-in incoming pulse of electromagnetic radiation (6). Due to the previous coupling in of high-energy electromagnetic radiation (8.0, 8.1), the effective refractive indices of the first, second, and fourth optical fibers (2.2) have reached the value of the effective refractive index of the third optical fiber, and the travel time differences of the exiting pulse of electromagnetic radiation (7) are compensated for.

FIG. 6 shows a schematic representation of the arrangement (4) suitable for measuring the travel time difference of image waveguides in at least one wavelength, having a light-emitting source of electromagnetic radiation (SLED) having a superluminescent diode, a Y-waveguide (Y 50:50), a first optical waveguide (SMF1), a second optical waveguide (SMF2+Lens) having a lens, a first biconvex lens (L1), a second biconvex lens (L2), a first beam splitter (BS1), a first mirror (M1), a second mirror (M2), a first polarization filter (PF1), a second polarization filter (PF2), a third polarization filter (PF3), a microscope lens (MO1), and an imaging detector for electromagnetic radiation (CAM) having a camera. Light is emitted by the source of electromagnetic radiation (SLED) and is coupled into the Y-waveguide (Y 50:50) and split. A first half of the light is coupled from the Y-waveguide (Y 50:50) into the first optical waveguide (SMF1) and coupled out of this waveguide in such a way that it first strikes the first biconvex lens (L1) and from the first biconvex lens (L1) strikes the first beam splitter (BS1) in such a way that the first beam splitter (BS1) transmits 50% of the first half of the light to the first mirror (M1), the first mirror (M1) reflects the 50% of the first half of the light to the first beam splitter (BS1), and the first beam splitter (BS1) reflects 50% of the 50% of the first half of the light through the first polarization filter (PF1) to the imaging detector for electromagnetic radiation (CAM) having a camera. A second half of the light is coupled from the Y-waveguide (Y 50:50) into the first end of the second optical waveguide (SMF2+Lens) having a lens at the second end, and is coupled out of this second waveguide at its second end. The second half of the light then passes through the second polarization filter (PF2), then through the third polarization filter (PF3), and is then coupled into an image waveguide (1, CFB) designed as a coherent bundle of optical fibers. The second half of the light is coupled out of the image waveguide (1, CFB) and passes through the microscope lens (MO1) and then the second biconvex lens (L2) to the second mirror (M2) which reflects it to the first beam splitter (BS1). The first beam splitter (BS1) transmits 50% of the second half of the light through the first polarization filter (PF1) to the imaging detector (CAM). The first mirror (M1) is arranged to be movable along the optical axis of the 50% of the first half of the light.

FIG. 7 shows a schematic representation of the device (3) for compensating for travel time differences and/or for implementing a desired travel time profile of the exemplary embodiment, having a light-emitting source of electromagnetic radiation (SLED) having a superluminescent diode, a Y-waveguide (Y 50:50), a first optical waveguide (SMF1), a second optical waveguide (SMF2+Lens) having a lens, a first biconvex lens (L1), a second biconvex lens (L2), a first beam splitter (BS1), a second beam splitter (BS2), a first mirror (M1), a first polarization filter (PF1), a second polarization filter (PF2), a third polarization filter (PF3), a microscope lens (MO1), an imaging detector for electromagnetic radiation (CAM) having a camera, a spatial light modulator (SLM), and a source of high-energy electromagnetic radiation (5). Light is emitted by the source of electromagnetic radiation (SLED) and is coupled into the Y-waveguide (Y 50:50) and split. A first half of the light is coupled from the Y-waveguide (Y 50:50) into the first optical waveguide (SMF1) and is coupled out of this waveguide in such a way that it first strikes the first biconvex lens (L1) and from the first biconvex lens (L1) strikes the first beam splitter (BS1) in such a way that the beam splitter (BS1) transmits 50% of the first half of the light to the first mirror (M1), the first mirror (M1) reflects the 50% of the first half of the light to the beam splitter (BS1) and the beam splitter (BS1) reflects 50% of the 50% of the first half of the light through the first polarization filter (PF1) to the imaging detector for electromagnetic radiation (CAM) having a camera. A second half of the light is coupled from the Y-waveguide (Y 50:50) into the first end of the second optical waveguide (SMF2+Lens) having a lens at the second end, and is coupled out of this second waveguide at its second end. The second half of the light then passes through the second polarization filter (PF2), then through the third polarization filter (PF3), and is then coupled into an image waveguide (1, CFB) designed as a coherent bundle of optical fibers. The second half of the light is coupled out of the image waveguide (1, CFB) and passes through the microscope lens (MO1) and then through the second biconvex lens (L2) to the second beam splitter (BS2) which reflects 50% of it to the first beam splitter (BS1). The beam splitter (BS1) transmits 50% of the 50% of the second half of the light through the first polarization filter (PF1) to the imaging detector (CAM). The first mirror (M1) is arranged to be movable along the optical axis of the 50% of the first half of the light.

FIG. 8 shows a schematic representation of the device (3) for compensating travel time differences and/or for implementing a desired travel time profile of the exemplary embodiment. High-energy electromagnetic radiation (8) is emitted by the source (5) and first passes through the spatial light modulator (SLM), then 50% thereof passes through the second beam splitter BS2, then through the second biconvex lens (L2) and through the microscope lens (MO1) into a first optical fiber of the image waveguide (1, CFB). The spatial light modulator (SLM) is set so that only the part of the high-energy electromagnetic radiation (8) which can be coupled into the first optical fiber is transmitted through the spatial light modulator (SLM). The part of the radiation (8) which would be coupled into other optical fibers of the image waveguide (1, CFB) in the absence of the spatial light modulator (SLM) is absorbed and/or reflected by the spatial light modulator (SLM).

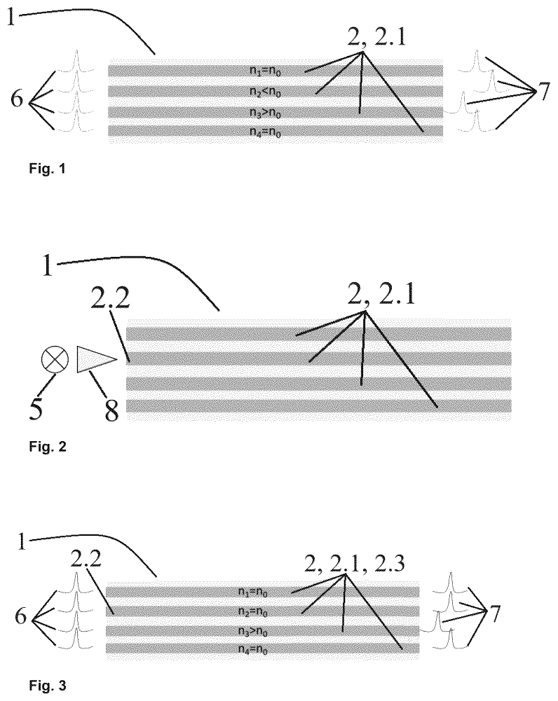

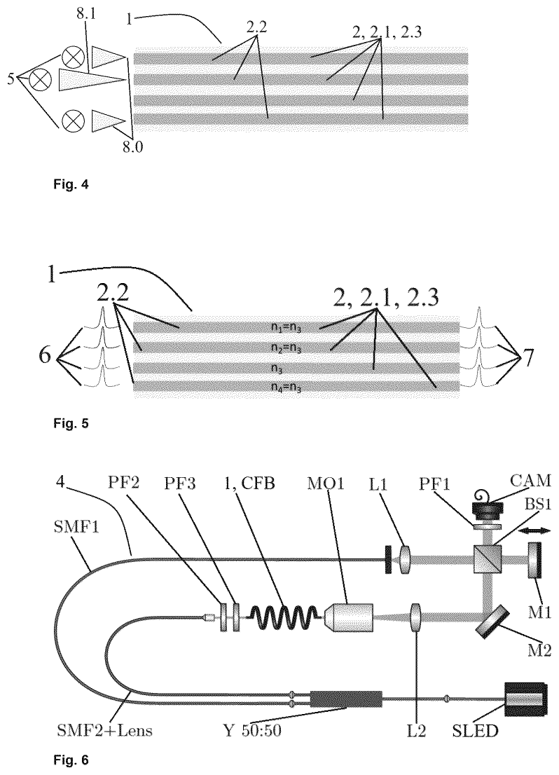

According to the invention, the object is achieved by a method and a device according to the independent claims. Advantageous embodiments of the invention are given in the dependent claims.

One aspect of the invention relates to a method for compensating for travel time differences and/or for implementing a desired travel time profile of at least one image waveguide having at least two optical fibers, comprising the steps of:
providing at least one image waveguide having at least two optical fibers,
selecting a first subset of at least two optical fibers of the image waveguide and measuring the travel time differences of the optical fibers of the first subset in at least one electromagnetic wavelength,
selecting a second subset of one or more optical fibers and a third subset of at least two optical fibers of the image waveguide, changing the effective refractive index of each of the optical fibers of the second subset by longitudinally coupling high-energy electromagnetic radiation into each of the optical fibers of the second subset at a first end and/or a second end of the image waveguide such that the travel time differences of the third subset of optical fibers are arbitrarily reduced, and/or such that the travel time differences of the third subset of optical fibers approach the value of the desired travel time profile as desired,
   wherein the second subset of optical fibers comprises at least one optical fiber of the first subset and the third subset of optical fibers comprises at least one optical fiber of the second subset, and the first subset of optical fibers also comprises this at least one optical fiber of the second subset, wherein the execution of the sequence of steps ii)-iii) takes place either once or as often as necessary until a desired compensation of the travel time differences and/or the desired travel time profile of the at least one image waveguide is implemented in the at least one wavelength, wherein when the sequence of steps ii)-iii) is repeatedly carried out, the subsets of optical fibers either each correspond to the respective subsets of optical fibers of the previous execution of the sequence of steps ii)-iii) or are newly selected, and wherein steps ii) and iii) take place sequentially or simultaneously.

In embodiments of the method:
the first subset of optical fibers comprises all the optical fibers of the image waveguide, and/or
the second subset of optical fibers comprises all the optical fibers of the first subset with the exception of those whose travel time difference from the reference travel time is already sufficiently small and/or already corresponds to the desired travel time profile, and/or
the third subset of optical fibers comprises all the optical fibers of the first subset.

The travel time differences of the optical fibers of the first subset from the reference travel time are measured individually in embodiments of the method.

In embodiments of the method, the high-energy radiation is coupled individually into the fibers of the second subset. As a rule, each optical fiber of the image waveguide has a different travel time, which is why each optical fiber requires a different change in the effective refractive index in order to sufficiently compensate for the travel time differences or to achieve the desired travel time profile. For this reason, the high-energy electromagnetic radiation is coupled into each optical fiber individually while being given different properties in each case, which in each case has the result that the effective refractive index of the particular optical fiber approximates the effective refractive index that is required for the respective desired travel time.

Steps ii) and iii) can be carried out simultaneously if the measurement of the travel time differences of the optical fibers is carried out using the same high-energy electromagnetic radiation which is suitable for changing the effective refractive index of the optical fibers.

In embodiments of the method, the high-energy electromagnetic radiation comprises ultra-short pulses and/or UV radiation, in particular femtosecond laser pulses and/or excimer light, wherein the excimer light advantageously contains 146 nm excimer light or 248 nm excimer light, and wherein the excimer light comprises excimer light and/or excimer laser light that can be emitted by excimer lamps.

The pulse duration of the ultra-short pulses can be between 10 fs and 10 ps in embodiments, and the pulse duration of the femtosecond laser pulses can be between 10 fs to 1 ps.

The term “light” includes, also as part of a term, the electromagnetic spectrum with wavelengths between 100 nm and 10 μm inclusive.

The optical fibers for which the method according to the invention is carried out are advantageously single-mode fibers. The cores of the optical fibers advantageously have a diameter that is small enough to transmit at most a single mode of electromagnetic radiation that is used to measure the travel time differences of the image waveguide and/or that is used to change the effective refractive indices of the optical fibers, but is large enough to prevent significant crosstalk>3 dB to adjacent optical fibers. This means that the diameter of the optical fibers is of the same order of magnitude as the diameter of the mode of electromagnetic radiation that is used to measure the travel time differences of the image waveguide and/or used to change the effective refractive indices of the optical fibers. Particularly advantageously, the diameter of the cores of the optical fibers is smaller than twice the diameter of the mode and larger than one-fifth of the diameter of the mode of the electromagnetic radiation that is used to measure the travel time differences of the image waveguide and/or used to change the effective refractive indices of the optical fibers.

A change in the effective refractive index of each of the selected optical fibers of the second subset of selected optical fibers is achieved in embodiments of the method by carrying out modulation of one or more correcting variables of the high-energy electromagnetic radiation, selected from the power, the energy, the pulse duration, the pulse shape, the spectral range, the spectral curve of the power, the temporal curve of the power, the spectral curve of the energy, the temporal curve of the energy, and the polarization.

For example, the effective refractive index of an optical fiber can be changed by coupling continuously emitted UV excimer light into the optical fiber
with low power over a long period of time, or
with high power over a short period of time.
   For example, it is also possible to change the effective refractive index of an optical fiber by coupling pulsed IR laser light or pulsed visible laser light with a pulse power of 10 MW into the optical fiber.

In embodiments of the method:
the at least one image waveguide is exposed, before and/or during the execution of the sequence of steps ii)-iii), to an atmosphere containing H₂ and/or N₂ in order to increase the partial pressure of the H₂ and/or N₂ inside the image waveguide, and/or
the selection of the modulation of the temporal and spectral curve of the radiation power of pulses of the high-energy electromagnetic radiation comprising ultra-short pulses is advantageously carried out in such a way that the radiation power integrated over the entire spectrum assumes a maximum value at a selected distance from the first end of the image waveguide within at least one of the selected optical fibers, and the modulation of the temporal and spectral curve of the radiation power of the high-energy electromagnetic radiation when the sequence of steps ii)-iii) is repeatedly carried out is particularly advantageously selected in such a way that, with each repetition, the radiation power assumes a maximum value at a distance other than that selected when the sequence of steps ii)-iii) was previously carried out.

Because the partial pressure of H₂ and/or N₂ in the image waveguide is increased compared to that of the earth's atmosphere, it is possible to achieve a greater change in the effective refractive index of the optical fibers by coupling high-energy electromagnetic radiation of a particular power, energy, a particular spectral range, spectral curve of the power, temporal curve of the power, spectral curve of the energy, temporal curve of the energy, and polarization into optical fibers of an image waveguide than would be the case with an identical image waveguide whose partial pressure of H₂ and/or N₂ is not increased.

A spatial change in the refractive index within an electromagnetic field, as is the case at an interface between two materials with different refractive indices, leads to electromagnetic energy being absorbed at this interface. When continuously emitted high-energy electromagnetic radiation is coupled into optical fibers of an image waveguide at one end, more energy is absorbed at that end of the optical fiber than in the rest of the fiber. This initially leads to a larger change in the effective refractive index than in the rest of the fiber and can, after continuous or repeated coupling in of the high-energy electromagnetic radiation, cause damage to the optical fiber in the region of the same end. Since the refractive index of a medium is wavelength-dependent and is inversely proportional to the propagation speed of electromagnetic radiation within the medium, the propagation speed depends on the wavelength.

If the propagation speed of electromagnetic radiation in a medium increases strictly monotonically with increasing wavelength, a broadband pulse of electromagnetic radiation would travel within the medium in such a way that a longer-wavelength part of the pulse traverses the medium first, followed by a shorter-wavelength part of the pulse. However, if a pulse is shaped so that short-wavelength radiation is coupled into the medium first, followed by longer-wavelength radiation, it is possible that the short-wavelength and long-wavelength radiation reach a region within the medium simultaneously, and the pulse power therefore reaches a maximum in this region and not at the interface region at which the radiation was coupled into the medium. By reducing or increasing the delay between short- and long-wavelength radiation of the emitted pulses, in each case a region closer to or further away from the interface region can be selected at which the pulse power is maximized. This can prevent the radiation power from always reaching its maximum value in the same region of a solid medium, and causing damage to the medium in this region, when ultrashort electromagnetic pulses are repeatedly coupled in.

Damage to the image waveguide in the region of the first end and/or the second end that can be caused by ultrashort pulses of high-energy electromagnetic radiation is kept to a minimum in embodiments of the methods:
by reducing the difference between the effective refractive indices of the fibers and the medium adjacent to the first end and/or the second end of the image waveguide during the execution of the method by
surrounding the first end and/or the second end of the image waveguide with an immersion liquid, advantageously an immersion oil, and/or
bringing at least one glass plate into contact with the first end and/or the second end of the image waveguide,
   wherein the materials of which the immersion liquid and/or the glass plate consist each comprise at least one material whose refractive index is arbitrarily close to the effective refractive index of at least one optical fiber of the second subset of selected optical fibers, and/or
by removing a part of the at least one image waveguide along a plane at the first end and/or at the second end after carrying out step iii), wherein the plane is perpendicular to the optical axis of the image waveguide and the length of the part to be removed or parts of the image waveguide to be removed along the optical axis corresponds or correspond to the length of the part or parts of the image waveguide which was or were destroyed by the longitudinal coupling in of high-energy electromagnetic radiation, and/or
by widening the cores of the optical fibers at the first end and/or at the second end of the image waveguide.

When the difference between the refractive indices of the optical fibers and the surrounding medium is small, the absorption of energy by the optical fibers at the interface between the optical fibers and the surrounding medium is also small compared to the absorption of energy by the optical fibers at the interface when the surrounding medium is air under standard conditions.

If the core of an optical fiber is widened at one of the ends of the image waveguide, the surface power density of the high-energy electromagnetic radiation coupled into the core of the optical fiber is lower than the surface power density of the same radiation in an optical fiber whose core is not widened at any end of the image waveguide. By widening the cores of the optical fibers at the first end and/or the second end of the image waveguide, the damage or ablation that is caused by the coupling in of the high-energy electromagnetic radiation is reduced. Widening an optical fiber at one end of the image waveguide can be achieved by heating, which diffuses dopants in the core of the optical fiber into the cladding of the optical fiber, thereby smearing the refractive index profile of the optical fiber at the heated end of the image waveguide.

A part of an image waveguide is considered damaged if the transmission in the wavelength range that is used to measure the travel time differences has decreased so much that the image waveguide can no longer be used for the desired purpose.

In embodiments of the method, the measurement of the travel time difference is carried out by means of white light interferometry and/or OCT and/or multi-wavelength holography.

In further embodiments, the multi-wavelength holography is designed as off-axis holography using a Mach-Zehnder interferometer.

In embodiments of the method, light is coupled into a Y-waveguide and split. A first part of the light is coupled into a provided image waveguide, is coupled from the image waveguide into a first magnifying lens and expanded, and reaches a beam splitter. The beam splitter transmits X % of the first part of the light to an imaging detector for electromagnetic radiation. A second part of the light is coupled out of the Y-waveguide in such as manner as to strike the beam splitter such that the beam splitter transmits X % of the second part of the light to a mirror, the mirror reflects the X % of the second part of the light to the beam splitter, and the beam splitter reflects (100−X) % of the X % of the second part of the light to the imaging detector.

The first magnifying lens and the imaging detector are arranged relative to each other and to the image waveguide and are each designed in such a way that structures, such as interference patterns on the facets of the optical fibers, can be resolved by the imaging detector. The mirror is moved along the optical axis of the X % of the second part of the light until one of the optical fibers exhibits an interference pattern from the perspective of the imaging detector. The travel time of the part of the light that is guided through the optical fiber exhibiting the interference pattern can be selected as the reference travel time. The mirror is moved further along the optical axis of the X % of the second part of the light until each optical fiber of the image waveguide for which a measurement of the travel time difference with respect to the reference time is desired has exhibited an interference pattern during the movement of the mirror. Upon each appearance of an interference pattern, the position of the mirror and of the optical fiber exhibiting the interference pattern is recorded on a storage medium. Based on the relative position of the mirror and the knowledge of the speed of light in the medium surrounding the mirror, the travel time difference with respect to the reference travel time is determined for each of the optical fibers that exhibit an interference pattern at a particular position of the mirror.

X can be any real number in the range 0<X<100. X=50 is advantageous. Advantageously, the first part and the second part of the light each amount to 50% of the part of the light coupled into the Y-waveguide. Advantageously, the light comprises light emitted by a superluminescent diode. Other optical components in the beam path, such as polarization filters, lenses, beam splitters and/or mirrors, are not excluded in the method.

High-energy electromagnetic radiation is coupled into individual optical fibers of the image waveguide through the first magnifying lens.

In embodiments, the first magnifying lens, the image waveguide as well as the source of high-energy electromagnetic radiation are positioned relative to one another such that in each case, the high-energy radiation can be coupled into a single optical fiber of the image waveguide. The relative position of the source of high-energy electromagnetic radiation, the image waveguide and the first magnifying lens can be changed after each coupling of the high-energy electromagnetic radiation into one of the optical fibers in such a way that the high-energy electromagnetic radiation can be coupled into another optical fiber of the image waveguide.

In embodiments, a spatial light modulator is arranged in the beam path of the high-energy electromagnetic radiation and adjusted in such a way that the high-energy radiation can in each case be coupled into a single optical fiber of the image waveguide. The setting of the spatial light modulator can be changed after each coupling of the high-energy electromagnetic radiation into one of the optical fibers in such a way that the high-energy electromagnetic radiation can be coupled into another optical fiber of the image waveguide.

In embodiments of the method, the functional relationships between the one or more correcting variables of the high-energy electromagnetic radiation and travel time changes of optical fibers are determined in each case by a calibration.

For example, for calibration, the travel times and/or the effective refractive indices of one or more optical fibers with optical and material properties and lengths that are similar to those of the image waveguide can be measured and subsequently or simultaneously exposed to high-energy electromagnetic radiation with particular correcting variables, and the travel times and/or the effective refractive indices can be measured again. This process can be repeated multiple times with one fiber each time. The correcting variables can then be entered into one or more calibration tables together with the associated changes in the travel times and/or effective refractive indices.

In addition to the correcting variables, travel times, and/or the effective refractive indices of optical fibers used for calibration, the changes in the transmission of the optical fibers used for calibration caused by exposure to high-energy electromagnetic radiation can also be measured and entered into the calibration table.

Other calibration methods are not excluded.

In embodiments of the method, after carrying out step iii), a method is executed for compensating for phase distortion of at least two wavelengths λₖ of the at least one image waveguide and/or for implementing at least one optical function which changes propagation directions of electromagnetic radiation of at least one wavelength λ_{ƒ} when entering and/or exiting the image waveguide, the method comprising the modulating of the electromagnetic phase distortion having a functional relationship with a reference path length φᵢₛ of a fifth subset of at least one optical fiber j that is selected from a fourth subset of two or more optical fibers of the image waveguide, for each of the wavelengths λₖ and/or λ_{ƒ}, comprising the sub-steps:
a) measuring the electromagnetic phase distortion φᵢₛ for each of the wavelengths λₖ and/or λ_{ƒ} on the optical fibers of the fourth subset,
b) determining a desired modulated phase φ_{des} for each of the fifth subset of selected optical fibers j and for each of the wavelengths λₖ and/or λ_{ƒ}, wherein the desired modulated phase φ_{des} for each of the wavelengths λₖ and/or λ_{ƒ} is determined independent of each other or depending on φ_{des} for one or more of the other wavelengths λₖ and/or λ_{ƒ},
c) determining a functional relationship between a correcting variable xⱼ and a phase change φₛₑₜ for each of the wavelengths λₖ and/or λ_{ƒ} and each of the selected optical fibers j of the fifth subset,
d) defining an error function ƒ to describe the overall deviation between a resulting phase φᵣₑₛ=(φᵢₛ+φₛₑₜ) mod (2π) and the desired modulated phase φ_{des} over all wavelengths λₖ and/or λ_{ƒ} for each of the selected optical fibers j of the fifth subset,
e) determining the value x_{j_ƒ min} of the correcting variable x; for which the error function ƒ assumes a minimum value for each of the selected optical fibers j of the fifth subset,
f)
   providing and positioning an element for compensating for phase distortion of at least two wavelengths λₖ of an image waveguide and/or for implementing an optical function which changes propagation directions of electromagnetic radiation of at least one wavelength λ_{ƒ} when entering and/or exiting the image waveguide, behind the first end and/or behind the second end of the image waveguide, such that the element along the optical axis of each of the selected optical fibers j of the fifth subset has the value x_{j_ƒ min} of the correcting variable xⱼ,
and/or
   shortening and/or lengthening each selected optical fiber j of the fifth subset to compensate for the phase distortion and/or to implement an optical function which changes propagation directions of electromagnetic radiation when entering and/or exiting the image waveguide, at the first end and/or at the second end of the image waveguide, such that the shortening and/or the lengthening for each of the selected optical fibers j of the fifth subset and each of the wavelengths λₖ and/or λ_{ƒ} has the value x_{j_ƒ min} of the correcting variable xⱼ,
so that the image waveguide having the element and/or the shortening and/or lengthening of each of the selected optical fibers j of the fifth subset for each of the wavelengths λₖ and/or λ_{ƒ} and each of the selected optical fibers j of the fifth subset has a resulting phase φ_{res_ƒ min} in which the error function ƒ assumes a minimal value.

In embodiments of the method:
the fourth subset of optical fibers comprises all the optical fibers of the image waveguide, and/or
the fifth subset of optical fibers comprises all optical fibers of the fourth subset with the exception of those whose phase distortion is already sufficiently low and/or already correspond to the desired optical function.

The set of wavelengths λₖ and the set of wavelengths λ_{ƒ} can be completely different, overlap with each other, or be identical.

The phase distortion φᵢₛ of an optical fiber with index j at one wavelength λ can be described using the formula

$${φ}_{i s} ( λ , j ) = 2 π ( \frac{Δ L ( λ , j )}{λ} mod 1 ) ,$$

where ΔL is a deviation of an optical path length of the optical fibers j at the wavelength λ from the average optical path length at the wavelength λ of all optical fibers of the fourth subset.

Other descriptions of the phase distortion φᵢₛ are not excluded. In an alternative embodiment, instead of ΔL, a reference path length can be used which is the deviation of the optical path length of an optical fiber from an arbitrary reference length.

The desired modulated phase φ_{des} can be set for each of the wavelengths independently or depending on the desired modulated phase φ_{des} for one or more of the other wavelengths, thus making it possible to implement different optical functions for different wavelengths. This means, for example, that for electromagnetic radiation of a first wavelength, a bundling of the radiation to a focal point is desired, while for radiation of a second wavelength, a donut mode is desired, and for radiation of a third wavelength, a tilting of the propagation direction is desired. Combinations of such optical functions, such as tilting the propagation direction and focusing the radiation of one wavelength on a focal point, are also possible. It is also possible that φ_{des} is selected for different wavelengths such that the propagation direction of the radiation is tilted by a different angle for each of the different wavelengths and/or is bundled at a different focal point for each of the different wavelengths. Examples of such optical functions are the focusing of the radiation to a focal point in a plane, similar to what is possible with a convex lens, the tilting of the radiation, or the generation of a donut mode, i.e. a ring-shaped distribution of the intensity of electromagnetic radiation in a plane.

In embodiments of the method for compensating for phase distortion and/or for implementing at least one optical function, the desired modulated phase φ_{des} can be described by the formula φ_{des}(λ, j)=(φᵢₛ(λ, j)+φ_{shift}(λ, j) mod (2π), where φ_{shift}(λ, j) is a desired phase shift.

A change in the optical path length which results in the desired modulated phase φ_{des} can be described using the formula

$${L}_{d e s} ( λ , j ) = \frac{{λφ}_{shift} ( λ , j )}{2 π} + N λ$$

where N is any integer.
In further embodiments of the method for compensating for phase distortion and/or for implementing at least one optical function, N lies in the range between −9 (inclusive) and +9 (inclusive).

In embodiments of the method for compensating for phase distortion and/or for implementing at least one optical function in which it is desired to compensate for the phase distortion φᵢₛ for one wavelength λ and an optical fiber j and the implementation of an additional optical function φ_{add} is also desired, the desired phase shift φ_{shift}(λ, j) can be determined by the formula

$${φ}_{shift} ( λ , j ) = ( ( - {φ}_{i s} ( λ , j ) ) + {φ}_{a d d} ( λ , j ) ) mod ( 2 π )$$

In further embodiments of the method for compensating for phase distortion and/or for implementing at least one optical function in which it is only desired to compensate for the phase distortion φᵢₛ for one wavelength λ and one optical fiber j without implementing an additional optical function, the additional optical function φ_{add}(λ, j)=0 can be used, whereby the desired phase shift described by the formula φ_{shift}(λ, j)=(−φᵢₛ(λ, j)) mod (2π), and the phase distortion φᵢₛ is fully compensated for.

Since the correcting variable xⱼ in general cannot be varied for each wavelength individually, it is generally not possible to achieve the ideal state φ_{des}=φᵣₑₛ for each wavelength and for each optical fiber of the fifth subset, which is why it is necessary to minimize the error function ƒ to get as close to the ideal state as possible.

In embodiments of the method for compensating for phase distortion and/or for implementing at least one optical function, the error function ƒ is determined
by taking a square root of the squares, summed over all of the wavelengths, of the deviation between the resulting phase φᵣₑₛ and the desired modulated phase φ_{des} or
by summing, over all the wavelengths, the amounts of the deviation between the resulting phase φᵣₑₛ and the desired modulated phase φ_{des}, for each of the optical fibers of the fifth subset.

The error function ƒ can be determined by the formula

$$f = \sqrt{{\sum }_{i = 1}^{i \geq 2} {( ( {φ}_{r e {s}_{i}} - {φ}_{d e {s}_{i}} ) mod ( 2 π ) )}^{2}}$$

or by means of the formula

$$f = {\sum }_{i = 1}^{i \geq 2} \vert ( {φ}_{r e {s}_{i}} - {φ}_{d e {s}_{i}} ) mod ( 2 π ) \vert$$

In this case, i is an index of one of the wavelengths, φ_{des}_{ᵢ} and φᵣₑₛ_{ᵢ} are in each case the desired modulated phase, and the resulting phase for the wavelength with index i and the expression i≥2 represents the number of the at least two wavelengths.

In embodiments of the method for compensating for phase distortion and/or for implementing at least one optical function, the resulting value of the correcting variable x_{j_ƒ min} is determined for each of the optical fibers of the fifth subset by an iterative method, wherein the error function ƒ is minimized by the iterative method. An advantage of iterative methods over computational methods is that the former are robust in relation to model errors, whereas the precision of computational methods is limited by the accuracy of the mathematical models on which they are based.

In embodiments of the method for compensating for phase distortion and/or for implementing at least one optical function, the resulting value of the correcting variable x_{j_ƒ min} is determined for each of the optical fibers of the fifth subset by an iterative method comprising the steps of:
a) measuring, in a plane behind the first end or behind the second end of the image waveguide or of the image waveguide having the element, the intensity of electromagnetic radiation guided through each of the optical fibers of the fourth subset in each of the wavelengths,
b) determining the difference between the measured intensity and the expected intensity for the desired modulated phase φ_{des}, for each of the wavelengths and each of the optical fibers of the fourth subset,
c) changing the value of the correcting variable x; for each of the optical fibers of the fifth subset,
d) carrying out steps a), b) and c) until a local minimum or the global minimum of the difference between the measured intensity and the intensity expected with the desired modulated phase φ_{des} is determined, and setting the correcting variable xⱼ to the value at which the determined local minimum or global minimum is reached, for each of the optical fibers of the fifth subset.

In embodiments of the method for compensating for phase distortion and/or for implementing at least one optical function, the correcting variable xⱼ has a functional relationship with
a) a path length difference ΔSⱼ and/or
b) a voltage Uⱼ and/or
c) an electrical current Iⱼ and/or
d) a current pulse width P_{Ij} and/or
e) a voltage pulse width P_{Uj} and/or
f) a temperature Tⱼ and/or
g) an SLM grayscale value.

The correcting variable xⱼ can be adjusted by means of different methods.

In embodiments, if it is adjusted by means of a shortening and/or lengthening of the optical fibers of the fifth subset, the correcting variable xⱼ has a functional relationship with a path length difference ΔSⱼ caused by the shortening and/or lengthening. The correcting variable xⱼ also has a functional relationship with a path length difference ΔSⱼ when it is adjusted by means of an additive or ablative manufacturing of a transmissive element.

In further embodiments, if the correcting variable xⱼ is adjusted by means of a spatial light modulator, it can have a functional relationship with one or more physical quantities with which the spatial light modulator is controlled. This quantity or quantities can be a voltage Uⱼ and/or an electrical current Iⱼ and/or a current pulse width P_{Ij} and/or a voltage pulse width P_{Uj} and/or a temperature Tⱼ. This quantity or these quantities can also have a functional relationship with a path length difference ΔSⱼ. This is the case, for example, if a spatial light modulator has an arrangement of micromirrors that can be separately controlled, lowered, raised and/or tilted.

The correcting variable x; can be controlled by current or voltage pulse width modulation in embodiments with spatial light modulators.

The correcting variable x; can be controlled in further embodiments with spatial light modulators by means of temperature modulation, wherein the temperature is functionally related to a current and/or a voltage. Thermo-optically modulated spatial light modulators, for example, can be controlled by temperature, which in turn can be controlled by an electrical current.

In embodiments, each element of a spatial light modulator can assume grayscale values in the range from 0 to 255.

The phase change φₛₜₑₗₗ of an optical fiber with index j at one wavelength λ can be described using the formula

$${φ}_{s e t} ( λ , j ) = \frac{{x}_{j} ( n ( λ , j ) - {n}_{U} ( λ ) )}{λ} mod ( 2 π ) ,$$

where n(λ, j) is the refractive index of the optical fiber j at the wavelength λ for the material to which the correcting variable xⱼ is applied, and n_{U}(λ) is the refractive index of the medium surrounding the image waveguide at the wavelength λ.

Other descriptions of the phase change φₛₜₑₗₗ are not excluded.

In general, different values of the correcting variable xⱼ can have the same phase change φₛₑₜ for one wavelength. This principle makes use of the method for compensating for phase distortion and/or for implementing at least one optical function to determine a value of the correcting variable x; at which the resulting phase φᵣₑₛ=(φᵢₛ+φₛₑₜ) mod (2π) corresponds as closely as possible to the desired modulated phase φ_{des} for all of the wavelengths. Surprisingly, this is the case with values of the correcting variable x; for which the phase change φₛₑₜ would be far beyond 2π if it did not comprise the modulo-operator mod (2π).

In embodiments of the method for compensating for phase distortion and/or for implementing at least one optical function, the functional relationships between the correcting variable xⱼ and the variables mentioned in a) to f) are each determined by a calibration, and/or the functional relationship between the correcting variable xⱼ and the path length change ΔSⱼ comprises the difference—normalized for the particular wavelength—between
a) the refractive index of the lengthened and/or shortened optical fibers and/or of the element, and
b) the refractive index of the medium surrounding the image waveguide.

If the correcting variable xⱼ is adjusted by a spatial light modulator comprising a micromirror arrangement, the phase change φₛₑₜ can be described for example by

$${φ}_{s e t} ( λ , j ) = 2 π ( \frac{{x}_{j}}{λ} mod 1 ) ,$$

where the correcting variable xⱼ with the path length difference ΔSⱼ comprises the functional relationship

$${x}_{j} = {n}_{U} ( λ ) Δ {S}_{j} \cdot \frac{{x}_{j}}{λ}$$

can contain values between and including −9 and +9.

If the correcting variable xⱼ is adjusted by shortening and/or lengthening the optical fibers of the fifth subset and/or by a transmissive element, the phase change φₛₑₜ can be described for example by

$${φ}_{s e t} ( λ , j ) = 2 π ( \frac{{x}_{j}}{λ} mod 1 ) ,$$

where the correcting variable x; with the path length difference ΔSⱼ comprises the functional relationship xⱼ=((n(λ,j)−n_{U}(λ))ΔSⱼ. Here

$$\frac{{x}_{j}}{λ}$$

can contain values between and including −9 and +9.

In embodiments of the method, the compensation of phase distortion and/or the implementation of an optical function is accomplished by a static element which is either a transmissive or a reflective element and/or by an adaptive element which is either a transmissive or a reflective element, wherein a reflective element is positioned at a distance from the particular end of the image waveguide behind which it is positioned, and images a phase mask onto the particular end of the image waveguide by reflecting electromagnetic radiation of the at least two wavelengths from a suitable angle of incidence.

An adaptive element is expediently designed as a spatial light modulator, wherein the spatial light modulator is an electro-optically modulated spatial light modulator, or a thermo-optically modulated spatial light modulator. Advantageously, the spatial light modulator is designed as an LCoS.

The distance of the reflective element from the particular end of the image waveguide behind which it is positioned can be chosen as desired. Advantageously, the distance lies within the range between 10,000 times the smallest of the wavelengths and 10,000,000 times the largest of the wavelengths, particularly advantageously within the range between 10,000 times the smallest of the wavelengths and 100,000 times the largest of the wavelengths.

The suitable angle of incidence of electromagnetic radiation on the reflective element is above 0° and below 90°, advantageously between 10° and 80°.

In embodiments,
the lengthening of the optical fibers of the fifth subset to compensate for phase distortion and/or to implement at least one optical function takes place by additive manufacturing to the optical fibers of the fifth subset at the first end and/or at the second end of the image waveguide and/or
the shortening of the optical fibers of the fifth subset to compensate for the phase distortion and/or to implement an optical function by laser ablation and/or by electron beam ablation of the optical fibers of the fifth subset at the first end and/or the second end of the image waveguide and/or
the provision of the element at the first end and/or at the second end of the image waveguide is effected by additive manufacturing on an element blank and/or by laser ablation and/or by electron beam ablation of an element blank, and/or
the provision of the element at the first end and/or the second end of the image waveguide is effected by manufacturing metaoptics, wherein the metaoptics are characterized in that they have structures whose dimensions are smaller than the smallest of the wavelengths.

Within the meaning of the method for compensating for phase distortion of at least two wavelengths λₖ of the at least one image waveguide and/or for the implementation of at least one optical function, an “element blank” refers to the element in its state temporally before the additive manufacturing and/or laser ablation and/or electron beam ablation, by means of which it is manufactured into an element for compensating for phase distortion of at least two wavelengths of an image waveguide and/or for implementing at least one optical function.

In embodiments of the method, additive manufacturing comprises one-photon polymerization and/or two-photon polymerization and/or multi-photon polymerization.

In embodiments of the method for compensating for phase distortion of at least two wavelengths λₖ of the at least one image waveguide and/or for implementing at least one optical function, sub-step b) i) of the method according to the invention is carried out either by means of white light interferometry or by means of digital holography and/or a phase retrieval method.

Digital holography and the phase retrieval method can be used together.

A further aspect of the invention relates to a device for compensating for travel time differences and/or for implementing a desired travel time profile of at least one image waveguide having at least two optical fibers, comprising an arrangement suitable for measuring the travel time difference of image waveguides in at least one wavelength, wherein
the arrangement suitable for measuring the travel time difference of the image waveguide comprises a source of high-energy electromagnetic radiation suitable for changing the effective refractive indices of optical fibers, and the source
can be used as a radiation source for measuring the travel time difference of the optical fibers and a simultaneous change in the effective refractive index of the optical fibers and/or
can be operated as a source of low-energy radiation by reducing the radiation power and/or by implementing an optical filter between the source and the image waveguide and can be used for measuring the travel time difference of the optical fibers and/or
the device comprises a source of high-energy radiation separate from the arrangement and suitable for changing the effective refractive indices of optical fibers,
   further comprising at least one first positioning device which is suitable
to position the image waveguide and the arrangement relative to each other in such a way as to enable the measurement of the travel time difference and/or the change of the effective refractive indices of optical fibers of the image waveguide and/or
to position the image waveguide and the source of high-energy electromagnetic radiation relative to one another in such a way that the longitudinal coupling of radiation emittable by the source into at least one optical fiber is possible.

The travel time differences of the optical fibers of the image waveguide from the reference travel time can be measured individually in embodiments of the device.

In embodiments of the device, the high-energy radiation is coupled into the fibers of the image waveguide individually and can be given different properties in each case during this, which in each case makes it possible for the effective refractive index of the particular optical fiber to approximate the effective refractive index that is required for the particular desired travel time.

In embodiments, the source of high-energy electromagnetic radiation comprises at least one ultra-short pulsed laser, and/or at least one UV light source, in particular at least one femtosecond laser and/or at least one excimer light source, wherein the excimer light source is advantageously a 146 nm excimer light source or a 248 nm excimer light source, and wherein the excimer light source comprises at least one excimer lamp and/or at least one excimer laser.

In embodiments, the ultra-short pulsed laser enables the emission of pulses with a pulse duration between 10 fs and 10 ps, and the femtosecond laser enables the emission of pulses with a pulse duration between 10 fs to 1 ps.

In embodiments of the device, the source of high-energy electromagnetic radiation is designed for modulation of one or more correcting variables of the radiation, selected from the power, the energy, the pulse duration, the pulse shape, the spectral range, the spectral curve of the power, the temporal curve of the power, the spectral curve of the energy, the temporal curve of the energy, and the polarization.

A continuously emitting UV excimer lamp is expediently designed, for example, to emit UV excimer light and to couple it into an optical fiber
with low power over a long period of time, or
with high power over a short period of time
   in order to change the effective refractive index of the optical fiber. It is also possible, for example, for a pulsed IR laser or a pulsed visible laser with a pulse power of 10 MW to be designed to emit pulsed IR laser light and to couple it into an optical fiber in order to change the effective refractive index of the optical fiber.

In embodiments, the device has at least one H₂ and/or N₂ chamber, wherein the H₂ and/or N₂ chamber comprises a gas container sealable in airtight fashion and a line connectable to the gas container for conducting H₂ and/or N₂ gas, wherein the line is connectable to a H₂ and/or N₂ gas network and/or to a pressure container suitable for containing H₂ and/or N₂ gas, and advantageously a device suitable for conveying the gas in the gas container out and/or a device suitable for conveying the H₂ and/or N₂ gas in, and wherein the chamber is designed to contain the at least one image waveguide, and the chamber advantageously has at least one region transparent for at least the half-width
of the radiation that can be used to measure the travel time difference and
of the high-energy radiation
   and the first positioning device is arranged in the chamber or the chamber has at least one second positioning device which is designed to position the image waveguide within the chamber such that the high-energy radiation and the radiation which can be used to measure the travel time difference can be coupled longitudinally into the image waveguide.

In embodiments of the device, the modulation of the temporal and spectral curve of the radiation power of the ultra-short pulsed laser can advantageously be designed such that the radiation power integrated over the entire spectrum assumes a maximum value at a selectable distance from the first end of the image waveguide within the at least one optical fiber when the radiation is coupled longitudinally into the at least one optical fiber of the image waveguide.

In embodiments, the device has
at least one apparatus which is designed to widen the cores of the optical fibers of the image waveguide at the first end and/or at the second end and/or
at least one liquid container suitable for containing an immersion liquid, in particular an immersion oil, which is designed to contain at least the first end and/or at least the second end of the at least one image waveguide and has at least one region transparent for at least the half-width of the spectral range
of the radiation that can be used to measure the travel time differences and
of the high-energy radiation
   and the first positioning device is arranged in the liquid container or the liquid container has at least one third positioning device which is designed to position at least the first end and/or at least the second end of the image waveguide within the chamber such that the radiation which can be used to measure the travel time difference and the high-energy radiation can be coupled longitudinally into the image waveguide and/or
at least one glass plate, wherein the glass plate and/or the image waveguide can be positioned such that the glass plate is in contact with the first end and/or the second end of the image waveguide,
   wherein the materials from which the immersion liquid and/or the glass plate are made each comprise at least one material whose refractive index is arbitrarily close to the effective refractive index of at least one optical fiber of the image waveguide and the material in each case is transparent at least for the half-width of the wavelength of the electromagnetic radiation that can be emitted and absorbed by the arrangement suitable for measuring the travel time difference of image waveguides.

The apparatus, which is designed to widen the cores of the optical fibers of the image waveguide at the first end and/or at the second end, can be a CO₂ laser which is designed to heat the first end and/or the second end of the image waveguide.

In embodiments of the device, the arrangement suitable for measuring the travel time difference of image waveguides in at least one wavelength comprises at least one white light interferometer and/or at least one optical coherence tomograph.

In further embodiments, the multi-wavelength holography is designed as off-axis holography using a Mach-Zehnder interferometer.

In embodiments, the device has at least one first source of electromagnetic radiation, at least one Y-waveguide, at least one first magnifying lens, at least one beam splitter, at least one mirror, and at least one imaging detector for electromagnetic radiation. If the first source of electromagnetic radiation cannot be designed as a source of high-energy electromagnetic radiation, the device further has a second source of electromagnetic radiation which can be designed as a source of high-energy electromagnetic radiation. The first source of electromagnetic radiation and the Y-waveguide can be positioned relative to one another such that electromagnetic radiation from the first source can be coupled into a first end of the Y-waveguide and can be split, and can be coupled out at a second end and a third end. The second end of the Y-waveguide and the first magnifying lens are designed such that a provided image waveguide can be positioned relative to the second end of the Y-waveguide and the first magnifying lens such that electromagnetic radiation can be coupled from the second end of the Y-waveguide into a first end of the image waveguide and can be coupled from a second end of the image waveguide into the first magnifying lens. The first magnifying lens and the beam splitter can be arranged relative to one another in such a way that electromagnetic radiation emerging from the first magnifying lens can strike the beam splitter, wherein the beam splitter is designed to transmit X % of the electromagnetic radiation striking it and to reflect (100−X) %. The beam splitter and the imaging detector can be positioned relative to each other in such a way that electromagnetic radiation exiting from the first magnifying lens and transmittable through the beam splitter can strike the imaging detector. The third end of the Y-waveguide, the beam splitter, and the mirror can be arranged relative to one another in such a way that X % of the electromagnetic radiation that can be coupled out of the third end of the Y-waveguide is transmittable by the beam splitter and can be reflected by the mirror to the beam splitter. The beam splitter and the imaging detector can be arranged in such a way that (100−X) % of the radiation that can be reflected by the mirror can be reflected by the beam splitter so that it strikes the imaging detector. The mirror can be positioned so as to be movable along the optical axis of the light that can be coupled out of the third end of the Y-waveguide and transmitted through the beam splitter so that the travel time of the electromagnetic radiation exiting from the third end of the Y-waveguide and reaching the imaging detector can be changed with respect to the travel time of the electromagnetic radiation exiting from the second end of the Y-waveguide and reaching the imaging detector. The first magnifying lens and the imaging detector are designed and can be arranged in relation to each other and to the image waveguide in such a way that structures such as interference patterns on the facets of the optical fibers can be resolved by the imaging detector.

X can be any real number in the range 0<X<100. Advantageously, the first part and the second part of the light each amount to 50% of the part of the light coupled into the Y-waveguide. X=50 is advantageous. Advantageously, the light comprises light emitted by a superluminescent diode. Further optical components in the beam path, such as polarization filters, lenses, beam splitters, and/or mirrors, are not excluded in embodiments of the device.

High-energy electromagnetic radiation can be coupled into individual optical fibers of the image waveguide by the first magnifying lens.

In embodiments, the source of high-energy electromagnetic radiation, the first magnification objective are designed and positionable relative to each other and to the image waveguide such that the high-energy radiation emitted by the first or second source can be coupled into individual optical fibers of the image waveguide and at individual optical fibers of the image waveguide. The relative position of the source of high-energy electromagnetic radiation, the image waveguide and the first magnification lens to each other and to the image waveguide can be changed such that the high-energy electromagnetic radiation can be coupled into further optical fibers of the image waveguide.

In embodiments, a spatial light modulator is arranged in the beam path of the high-energy electromagnetic radiation and is adjustable in such a way that the high-energy radiation of the source can in each case be coupled into a single optical fiber of the image waveguide. The setting of the spatial light modulator can be changed so that the high-energy electromagnetic radiation can be coupled into further optical fibers of the image waveguide.

In embodiments, the device has an apparatus for compensating for electromagnetic phase distortion of at least two wavelengths λₖ of the at least one image waveguide and/or for implementing a function which changes directions of propagation of electromagnetic radiation of at least one wavelength λ_{ƒ} when entering and/or exiting the image waveguide, comprising an arrangement suitable for measuring the phase distortion of image waveguides in at least two wavelengths, further comprising
an element which is suitable for compensating for electromagnetic phase distortion of at least two wavelengths λₖ and/or for implementing a function which changes propagation directions of electromagnetic radiation of at least one wavelength λ_{ƒ} when entering and/or exiting the image waveguide, wherein the element is positionable at a first end and/or a second end of the image waveguide and is modulated or modulable such that the element has a correcting variable along the electromagnetic propagation direction of one or more selected waveguides x_{j_ƒ min} and/or
a device suitable for shortening and/or lengthening optical fibers of image waveguides, wherein the device, wherein the image waveguide and the device can be positioned relative to one another in such a way that shortening and/or lengthening of optical fibers of the image waveguide is possible, such that optical fibers subjected to shortening and/or lengthening have a correcting variable x_{j_ƒ min},
   wherein the correcting variable x_{j_ƒ min} by carrying out the substeps a) to f) of the method for compensating for phase distortion of at least two wavelengths λₖ of the at least one image waveguide and/or for implementing at least one optical function which changes propagation directions of electromagnetic radiation of at least one wavelength λ_{ƒ} when entering and/or exiting the image waveguide.

In embodiments of the apparatus, the desired modulated phase odes can be described by the formula φ_{des}(λ, j)=(φᵢₛ(λ,j)+φ_{shift} (λ, j)) mod (2π), wherein φ_{shift}(λ, j) is a desired phase shift.

A change in the electromagnetic path length which results in the desired modulated phase φ_{des} can be calculated using the formula

$${L}_{d e s} ( λ , j ) = \frac{{λφ}_{shift} ( λ , j )}{2 π} + N λ$$

where N is any integer.

In further embodiments of the apparatus, N lies in the region between and including −9 and +9.

In embodiments of the apparatus that are designed to compensate for the phase distortion φᵢₛ for one wavelength λ and one waveguide j and also to implement an additional function φ_{add}, the desired phase shift φ_{shift}(λ, j) can be calculated by the formula

$${φ}_{shift} ( λ , j ) = ( - {φ}_{i s} ( λ , j ) + {φ}_{a d d} ( λ , j ) ) mod ( 2 π )$$

In further embodiments of the apparatus designed to compensate for the phase distortion φᵢₛ for one wavelength λ and one waveguide j without implementing an additional function, the additional function φ_{add}(λ, j)=0 can be used, such that the desired phase shift is determined by the formula φ_{shift}(λ,j)=(−φᵢₛ(λ, j)) mod (2π), and the phase distortion φᵢₛ is fully compensated for.

In embodiments of the apparatus, the correcting variable xⱼ has a functional relationship with
a) a path length difference ΔSⱼ and/or
b) a voltage Uⱼ and/or
c) an electrical current Iⱼ and/or
d) a current pulse width P_{Ij} and/or
e) a voltage pulse width P_{Uj} and/or
f) a temperature Tⱼ and/or
g) an SLM grayscale value.

In general, different values of the correcting variable xⱼ can have the same phase change φₛₑₜ for one wavelength. This principle makes use of the apparatus according to the invention to determine a value of the correcting variable x; at which the resulting phase φᵣₑₛ=(φᵢₛ+φₛₑₜ) mod (2π) corresponds to the desired modulated phase φ_{des} for all of the wavelengths as closely as possible. Surprisingly, this is the case with values of the correcting variable xⱼ for which the phase change φₛₑₜ would lie far beyond 2π if it did not comprise the modulo-operator mod (2π).

In embodiments of the apparatus comprising an element, the element is a static element which is either a transmissive or a reflective element and/or an adaptive element which is either a transmissive or a reflective element, wherein a reflective element is positioned at a distance from the corresponding end of the arrangement behind which it is positioned, and images a phase mask onto the corresponding end of the arrangement by reflecting electromagnetic radiation of the at least two wavelengths from a suitable angle of incidence, wherein the adaptive element is designed as a spatial light modulator.

In embodiments of the apparatus comprising an element, the element is a static element, the element having a path length difference ΔSⱼ along the electromagnetic propagation direction of each of the selected waveguides, with respect to a reference length. In this case, any reference length can be chosen. The path length difference ΔSⱼ for each of the selected waveguides is realized by surface properties of the element designed as a phase mask.

In embodiments of the apparatus, the material of the element for compensating for the phase distortion of the selected waveguides at the first end and/or at the second end of the corresponding waveguide comprises metaoptics, wherein the metaoptics are characterized in that they have structures whose dimensions are smaller than the smallest of the wavelengths.

Possible materials for optical fiber cores include silicon dioxide, chalcogenides, fluoride glasses, fluorozirconates, fluoroaluminates, phosphate glasses, corundum, polycarbonate, and/or polymethyl methacrylate. Other materials are not excluded.

Possible wavelengths include the spectral range between 100 nm and 10 μm, advantageously between 140 nm and 3 μm. Other spectral ranges are not excluded.

A further aspect of the invention relates to the use of
the method according to the invention and/or its embodiments and/or of
the device according to the invention and/or its embodiments
   for compensating for travel time differences and/or for implementing a desired travel time profile of an image waveguide having at least two optical fibers in cancer diagnostics, nonlinear endomicroscopy, OCT, swept-source OCT, for the undistorted transmission of femtosecond pulses, and/or for correcting travel time differences that occur in image waveguides having optical fibers twisted with each other.

The invention is not limited to the illustrated and described embodiments, but also includes all embodiments which act identically within the meaning of the invention. Furthermore, the invention is also not limited to the specifically described feature combinations, but may also be defined by any other combination of particular features of all individual features disclosed overall, provided the individual features are not mutually exclusive, or a specific combination of individual features is not explicitly excluded.

### EXEMPLARY EMBODIMENT

The invention is to be explained in more detail below on the basis of an embodiment. The exemplary embodiment relates to an embodiment of the method and the device according to the invention, and is intended to describe the invention without limiting it.

An image waveguide (1, CFB) designed as a coherent bundle of optical fibers is provided.

Light is emitted by a source of electromagnetic radiation having a superluminescent diode (SLED) and is coupled into a Y-waveguide (Y 50:50) and is split.

A first half of the light is coupled out from the Y-waveguide (Y 50:50) into a first optical waveguide (SMF1) and is coupled out of this waveguide in such a way that it first strikes a first biconvex lens (L1) and from the first biconvex lens (L1) strikes a first beam splitter (BS1) in such a way that the first beam splitter (BS1) transmits 50% of the first half of the light to a first mirror (M1), the first mirror (M1) reflects the 50% of the first half of the light to the first beam splitter (BS1) and the first beam splitter (BS1) reflects 50% of the 50% of the first half of the light through a first polarization filter (PF1) to an imaging detector for electromagnetic radiation (CAM) having a camera.

A second half of the light is coupled from the Y-waveguide (Y 50:50) into the first end of a second optical waveguide (SMF2+Lens) which has a lens at the second end, and is coupled out of this waveguide at its second end. The second half of the light then passes through a second polarization filter (PF2), then through a third polarization filter (PF3), and is then coupled into an image waveguide (1, CFB) designed as a coherent bundle of optical fibers. The second half of the light is coupled out of the image waveguide (1, CFB) and passes through a microscope lens (MO1) and then through a second biconvex lens (L2) to a second beam splitter (BS2) which reflects 50% of the second half of the light to the first beam splitter (BS1). The first beam splitter (BS1) transmits 50% of the 50% of the second half of the light through the first polarization filter (PF1) to the imaging detector (CAM).

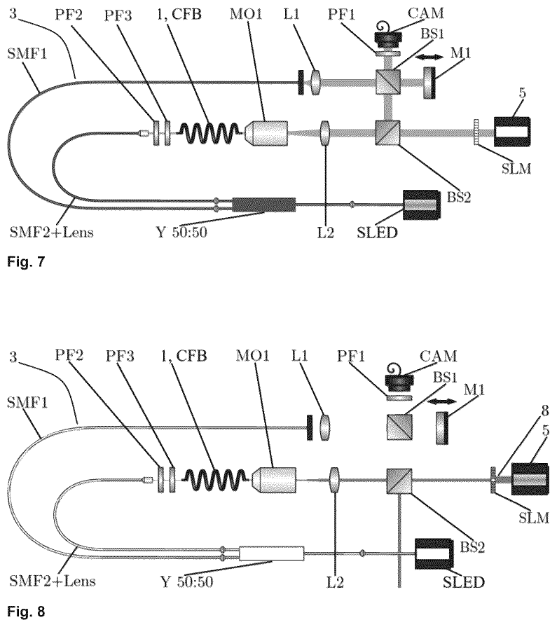

The image waveguide (1, CFB), the microscope lens (MO1), the second biconvex lens (L2), the second beam splitter (BS2), the first beam splitter (BS1), the first polarization filter (PF1), and the imaging detector (CAM) are arranged relative to one another and are each selected such that structures such as interference patterns on the facets of the optical fibers can be resolved by the imaging detector (CAM). The first mirror (M1) is moved along the optical axis of the 50% of the first half of the light until one of the optical fibers exhibits an interference pattern from the perspective of the imaging detector (CAM). The travel time of the part of the light that is guided through the optical fiber exhibiting the interference pattern can be selected as the reference travel time. The first mirror (M1) is moved further along the optical axis of the 50% of the first half of the light until each optical fiber of the image waveguide (1, CFB) of which a measurement of the travel time difference with respect to the reference travel time is desired has exhibited an interference pattern in the course of the movement of the first mirror (M1). Upon each appearance of an interference pattern, the position of the first mirror (M1) and of the optical fiber exhibiting the interference pattern is recorded on a storage medium. Based on the relative position of the first mirror (M1) and the knowledge of the speed of light in air, the travel time difference with respect to the reference travel time is determined for each of the optical fibers which exhibit an interference pattern at a particular position of the first mirror (M1). This configuration of the device (3) is shown schematically in FIG. 7.

High-energy radiation (8) with a surface power density of 4.2 mW/cm² emitted by a source of high-energy electromagnetic radiation (5) in the form of a 248-nm excimer lamp is coupled over a period of 10 minutes first through a spatial light modulator (SLM), then 50% through the second beam splitter BS2, then through the second biconvex lens (L2) and through the microscope lens (MO1) into a first optical fiber of the image waveguide (1, CFB). The spatial light modulator (SLM) is set so that only the part of the high-energy electromagnetic radiation (8) which can be coupled into the first optical fiber is transmitted through the spatial light modulator (SLM). The part of the radiation (8) which would be coupled into other optical fibers of the image waveguide (1, CFB) in the absence of the spatial light modulator (SLM) is absorbed and/or reflected by the spatial light modulator (SLM). Subsequently, the travel time of the first optical fiber is determined, and an increase in the effective refractive index of the first optical fiber by a factor of the order of 0.001 is determined. The setting of the spatial light modulator (SLM) is then changed so that the high-energy electromagnetic radiation can be coupled into another optical fiber of the image waveguide (1, CFB). This configuration of the device (3) is shown schematically in FIG. 8.

### CITED NON-PATENT LITERATURE

[1] Yoshinari Maezono et al., “Study of Refractive Index Change in Ge-Doped Fibers with Vacuum Ultraviolet Light Irradiation,” 2008, Jpn. J. Appl. Phys. 47 7266
[2] Lancry et al., “Dependence of the femtosecond laser refractive index change thresholds on the chemical composition of doped-silica glasses,” 2011, Opt. Mater. Express 1, 711-723
[3] Mirsky, S. K., Shaked, N. T., “Six-pack holography for dynamic profiling of thick and extended objects by simultaneous three-wavelength phase unwrapping with doubled field of view,” 2023, Sci Rep 13, 19293

### REFERENCE SIGNS

1 Image waveguide
2 Optical fibers
2.1 First subset of optical fibers
2.2 Second subset of optical fibers
2.3 Third subset of optical fibers
2.4 Fourth subset of optical fibers
2.5 Fifth subset of optical fibers
3 Device for compensating for travel time differences
4 Arrangement suitable for measuring the travel time difference of the image waveguide
5 Source of high-energy electromagnetic radiation
6 Incoming pulse of electromagnetic radiation
7 Exiting pulse of electromagnetic radiation
8 High-energy electromagnetic radiation
8.0 High-energy electromagnetic radiation having lower energy
8.1 High-energy electromagnetic radiation having higher energy
SLED A source of electromagnetic radiation having a superluminescent diode
Y 50:50 Y-waveguide
SMF1 First optical waveguide
SMF2+Lens Second optical waveguide having a lens
L1 First biconvex lens
L2 Second biconvex lens
BS1 First beam splitter
BS2 Second beam splitter
M1 First mirror
M2 Second mirror
PF1 First polarization filter
PF2 Second polarization filter
PF3 Third polarization filter
MO1 Microscope lens
CAM Imaging detector for electromagnetic radiation having a camera
CFB Coherent bundle of optical fibers
SLM Spatial light modulator

1. A method for compensating for travel time differences and/or for implementing a desired travel time profile of at least one image waveguide (1) having at least two optical fibers (2), comprising the steps of:
providing at least one image waveguide (1) having at least two optical fibers (2),
selecting a first subset of at least two optical fibers (2.1) of the image waveguide (1) and measuring the travel time differences of the optical fibers of the first subset (2.1) in at least one electromagnetic wavelength,
selecting a second subset of one or more optical fibers (2.2) and a third subset of at least two optical fibers (2.3) of the image waveguide (1), changing the effective refractive index of each of the optical fibers of the second subset (2.2) by longitudinally coupling high-energy electromagnetic radiation into each of the optical fibers of the second subset (2.2) at a first end and/or a second end of the image waveguide (1) such that the travel time differences of the third subset of optical fibers (2.3) are arbitrarily reduced, and/or such that the travel time differences of the third subset of optical fibers (2.3) approach the value of the desired travel time profile as desired,
wherein the second subset of optical fibers (2.2) comprises at least one optical fiber of the first subset (2.1) and the third subset of optical fibers (2.3) comprises at least one optical fiber of the second subset (2.2), and the first subset of optical fibers (2.1) also comprises this at least one optical fiber of the second subset (2.2),
wherein the execution of the step sequence ii)-iii) is carried out either once or as often as necessary until a desired compensation of the travel time differences and/or the desired travel time profile of the at least one image waveguide (1) is implemented in the at least one wavelength, wherein when the step sequence ii)-iii) is carried out repeatedly, the subsets of optical fibers (2.1, 2.2, and 2.3) are either each the same as the corresponding subsets of optical fibers (2.1, 2.2, and 2.3) of the previous execution of the step sequence ii)-iii) or are newly selected, and
wherein steps ii) and iii) occur sequentially or simultaneously.

2. The method according to claim 1, characterized in that the high-energy electromagnetic radiation comprises ultra-short pulses and/or UV radiation, in particular femtosecond laser pulses and/or excimer light, wherein the excimer light advantageously contains 146 nm excimer light or 248 nm excimer light, wherein the excimer light comprises the excimer light and/or excimer laser light that can be emitted by excimer lamps.

3. The method according to claim 1, characterized in that a desired change in the effective refractive index of each of the optical fibers of the second subset (2.2) is achieved by modulating one or more correcting variables of the high-energy electromagnetic radiation, selected from the power, the energy, the pulse duration, the pulse shape, the spectral range, the spectral curve of the power, the temporal curve of the power, the spectral curve of the energy, the temporal curve of the energy, and the polarization.

4. The method according to claim 1,
characterized in that the at least one image waveguide (1) is exposed to an atmosphere containing H₂ or N₂ before and/or during the execution of the sequence of steps ii)-iii) in order to increase the H₂ or N₂ partial pressure inside the image waveguide (1) and/or
characterized by selecting the modulation of the temporal and spectral curve of the radiation power of pulses of the high-energy electromagnetic radiation comprising ultra-short pulses, advantageously in such a way that the radiation power integrated over the entire spectrum assumes a maximum value at a selected distance from the first end of the image waveguide (1) within at least one of the selected optical fibers, and the modulation of the temporal and spectral curve of the radiation power of the high-energy electromagnetic radiation during each repeated execution of the sequence of steps ii)-iii) is particularly advantageously selected such that during each repetition the radiation power assumes a maximum value at a different distance from the first end of the image waveguide than the distance selected during the previous execution of the sequence of steps ii)-iii).

5. The method according to claim 1, characterized in that the method is suitable for minimizing damage to the image waveguide (1) in the region of the first end and/or the second end that can be caused by ultrashort pulses of high-energy electromagnetic radiation
by reducing the difference between the effective refractive indices of the fibers and the medium adjacent to the first end and/or the second end of the image waveguide (1) during the execution of the method by
surrounding the first end and/or the second end of the image waveguide (1) with an immersion liquid, advantageously an immersion oil and/or
bringing at least one glass plate into contact with the first end and/or the second end of the image waveguide (1),
wherein the materials of which the immersion liquid and/or the glass plate consist each comprise at least one material whose refractive index is arbitrarily close to the effective refractive index of at least one optical fiber of the second subset of selected optical fibers (2.2) and/or
by removing a part of the at least one image waveguide (1) along a plane at the first end and/or at the second end after carrying out step iii), wherein the plane is perpendicular to the optical axis of the image waveguide (1) and the length of the part or parts of the image waveguide (1) to be removed along the optical axis corresponds or correspond to the length of the part or parts of the image waveguide (1) which was or were damaged by an absorption of at least part of the high-energy electromagnetic radiation and/or
by expanding the cores of the optical fibers at the first end and/or at the second end of the image waveguide (1).

6. The method according to claim 1, characterized in that the measurement of the travel time difference is carried out by means of white light interferometry and/or OCT and/or multi-wavelength holography.

7. The method according to claim 1, characterized in that after carrying out step iii), a method is executed for compensating for phase distortion of at least two wavelengths λₖ of the at least one image waveguide (1) and/or for implementing at least one optical function which changes propagation directions of electromagnetic radiation of at least one wavelength λ_{ƒ} when entering and/or exiting the image waveguide (1), the method comprising the modulating of the electromagnetic phase distortion having a functional relationship with a reference path length φᵢₛ of a fifth subset of at least one optical fiber j (2.5) that is selected from a fourth subset of two or more optical fibers (2.4) of the image waveguide (1), for each of the wavelengths λₖ and/or λ_{ƒ}, comprising the sub-steps
a) measuring the electromagnetic phase distortion φᵢₛ for each of the wavelengths λₖ and/or λ_{ƒ} on the optical fibers (2.4) of the fourth subset,
b) determining a desired modulated phase φ_{des} for each of the fifth subset of selected optical fibers j (2.5) and for each of the wavelengths λₖ and/or λ_{ƒ}, wherein the desired modulated phase Odes for each of the wavelengths λₖ and/or λ_{ƒ} is determined independent of each other or depending on φ_{des} for one or more of the other wavelengths λₖ and/or λ_{ƒ},
c) determining a functional relationship between a correcting variable xⱼ and a phase change (set for each of the wavelengths λₖ and/or λ_{ƒ} and each of the selected optical fibers j of the fifth subset (2.5),
d) defining an error function ƒ to describe the overall deviation between a resulting phase φᵣₑₛ=(φᵢₛ+φₛₑₜ) mod (2π) and the desired modulated phase φ_{des} over all wavelengths λₖ and/or λ_{ƒ} for each of the selected optical fibers j of the fifth subset (2.5),
e) determining the value x_{j_ƒ min} of the correcting variable xⱼ for which the error function ƒ assumes a minimum value for each of the selected optical fibers j of the fifth subset (2.5),
f) providing and positioning an element for compensating for phase distortion of at least two wavelengths λₖ of an image waveguide (1) and/or for implementing at least one optical function which changes propagation directions of electromagnetic radiation of at least one wavelength λ_{ƒ} when entering and/or exiting the image waveguide (1), behind the first end and/or behind the second end of the image waveguide (1), such that the element along the optical axis of each of the selected optical fibers j of the fifth subset (2.5) has the value x_{j_ƒ min} of the correcting variable xⱼ,
and/or
shortening and/or lengthening each selected optical fiber j of the fifth subset (2.5) to compensate for the phase distortion and/or to implement a function which changes propagation directions of electromagnetic radiation when entering and/or exiting the image waveguide (1), at the first end and/or at the second end of the image waveguide (1), such that the shortening and/or the lengthening for each of the selected optical fibers j of the fifth subset (2.5) and each of the wavelengths λₖ and/or λ_{ƒ} has the value x_{j_fmin} of the correcting variable xⱼ,
so that the image waveguide (1) comprising the element and/or the shortening and/or lengthening of each of the selected optical fibers j of the fifth subset (2.5) for each of the wavelengths λₖ and/or λ_{ƒ} and each of the selected optical fibers j of the fifth subset (2.5) has a resulting phase φ_{res_fmin} in which the error function ƒ assumes a minimal value.

8. A device (3) for compensating for travel time differences and/or for implementing a desired travel time profile of at least one image waveguide (1) having at least two optical fibers (2), comprising an arrangement (4) suitable for measuring the travel time difference of image waveguides in at least one wavelength, wherein
the arrangement (4) suitable for measuring the travel time difference of the image waveguide (1) comprises a source of high-energy electromagnetic radiation (5) suitable for changing the effective refractive indices of optical fibers, and the source (5)
can be used as a radiation source for measuring the travel time difference of the optical fibers and a simultaneous change in the effective refractive index of the optical fibers and/or
can be operated as a source of low-energy radiation by reducing the radiation power and/or by implementing an optical filter between the source (5) and the image waveguide (1) and can be used for measuring the travel time difference of the optical fibers
and/or
the device (3) comprises a source (5) of high-energy radiation separate from the arrangement (4) and suitable for changing the effective refractive indices of optical fibers,
further comprising at least one first positioning device which is suitable
to position the image waveguide (1) and the arrangement (4) relative to each other in such a way as to enable the measurement of the travel time difference and/or the change of the effective refractive indices of optical fibers of the image waveguide (1) and/or
to position the image waveguide (1) and the source of high-energy electromagnetic radiation (5) relative to one another in such a way that the longitudinal coupling of radiation emittable by the source (5) into at least one optical fiber is possible.

9. The device (3) according to claim 8, characterized in that the source of high-energy electromagnetic radiation comprises at least one ultra-short pulsed laser, and/or at least one UV light source, in particular at least one femtosecond laser and/or at least one excimer light source, wherein the excimer light source is advantageously a 146 nm excimer light source or a 248 nm excimer light source, and wherein the excimer light source comprises at least one excimer lamp and/or at least one excimer laser.

10. The device (3) according to claim 8, characterized in that the source of high-energy electromagnetic radiation is designed for modulation of one or more correcting variables of the radiation, selected from the power, the energy, the pulse duration, the pulse shape, the spectral range, the spectral curve of the power, the temporal curve of the power, the spectral curve of the energy, the temporal curve of the energy, and the polarization.

11. The device (3) according to claim 8,
characterized in that it comprises at least one H₂ and/or N₂ chamber, wherein the H₂ and/or N₂ chamber comprises a gas container sealable in airtight fashion and a line connectable to the gas container for conducting H₂ and/or N₂ gas, wherein the line is connectable to a H₂ and/or N₂ gas network and/or to a pressure container suitable for containing H₂ and/or N₂ gas, and advantageously a device suitable for conveying the gas in the gas container out and/or a device suitable for conveying the H₂ and/or N₂ gas in, and wherein the chamber is designed to contain the at least one image waveguide (1) and the chamber advantageously has at least one region transparent for at least the half-width
of the radiation that can be used to measure the travel time difference and
of the high-energy radiation
and the first positioning device is arranged in the chamber, or the chamber has at least one second positioning device which is designed to position the image waveguide (1) within the chamber such that the high-energy radiation and the radiation which can be used to measure the travel time difference can be coupled longitudinally into the image waveguide (1) and/or
characterized in that the modulation of the temporal and spectral curve of the radiation power of the ultra-short pulsed laser can advantageously be designed such that the radiation power integrated over the entire spectrum assumes a maximum value at a selectable distance from the first end of the image waveguide (1) within the at least one optical fiber when the radiation is coupled longitudinally into the at least one optical fiber of the image waveguide (1).

12. The device (3) according to claim 8, characterized in that
the device (3) has at least one apparatus which is designed to widen the cores of the optical fibers of the image waveguide (1) at the first end and/or at the second end, and/or
the device (3) has at least one liquid container suitable for containing an immersion liquid, in particular an immersion oil, which is designed to contain at least the first end and/or at least the second end of the at least one image waveguide (1) and has at least one region transparent for at least the half-width of the spectral range
of the radiation that can be used to measure the travel time differences and
of the high-energy radiation
and the first positioning device is arranged in the liquid container or the liquid container has at least one third positioning device which is designed to position at least the first end and/or at least the second end of the image waveguide (1) within the chamber such that the radiation which can be used to measure the travel time difference and the high-energy radiation can be coupled longitudinally into the image waveguide (1), and/or
the device (3) comprises at least one glass plate, wherein the glass plate and/or the image waveguide (1) can be positioned such that the glass plate is in contact with the first end and/or the second end of the image waveguide (1),
wherein the materials from which the immersion liquid and/or the glass plate are made each comprise at least one material whose refractive index is arbitrarily close to the effective refractive index of at least one optical fiber of the image waveguide (1) and the material in each case is transparent at least for the half-width of the wavelength of the electromagnetic radiation that can be emitted and absorbed by the arrangement (4) suitable for measuring the travel time difference of image waveguides.

13. The device (3) according to claim 8, characterized in that the arrangement (4) suitable for measuring the travel time difference of image waveguides in at least one wavelength comprises at least one white light interferometer and/or at least one optical coherence tomograph.

14. The device (3) according to claim 8, characterized in that it has an apparatus for compensating for electromagnetic phase distortion of at least two wavelengths λₖ of the at least one image waveguide (1) and/or for implementing a function which changes propagation directions of electromagnetic radiation of at least one wavelength λ_{ƒ} when entering and/or exiting the image waveguide (1), comprising an arrangement (4) suitable for measuring the phase distortion of image waveguides in at least two wavelengths, further comprising
an element which is suitable for compensating for electromagnetic phase distortion of at least two wavelengths λₖ and/or to implement a function which changes propagation directions of electromagnetic radiation of at least one wavelength λ_{ƒ} when entering and/or exiting the image waveguide (1), wherein the element is positionable at a first end and/or a second end of the image waveguide (1) and is modulated or can be modulated such that the element has a correcting variable along the electromagnetic propagation direction of one or more selected waveguides x_{j_ƒ min} and/or
a device suitable for shortening and/or lengthening optical fibers of image waveguides, wherein the device, wherein the image waveguide (1) and the device can be positioned relative to one another in such a way that shortening and/or lengthening of optical fibers of the image waveguide (1) is possible, such that optical fibers subjected to shortening and/or lengthening have a correcting variable x_{j_fmin},
wherein the correcting variable x_{j_ƒ min} can be determined by carrying out substeps a) to f):
a) measuring the electromagnetic phase distortion φᵢₛ for each of the wavelengths λₖ and/or λ_{ƒ} on the optical fibers (2.4) of the fourth subset,
b) determining a desired modulated phase φ_{des} for each of the fifth subset of selected optical fibers j (2.5) and for each of the wavelengths λₖ and/or λ_{ƒ}, wherein the desired modulated phase φ_{des} for each of the wavelengths λₖ and/or λ_{ƒ} is determined independent of each other or depending on φ_{des} for one or more of the other wavelengths λₖ and/or λ_{ƒ},
c) determining a functional relationship between a correcting variable xⱼ and a phase change (set for each of the wavelengths λₖ and/or λ_{ƒ} and each of the selected optical fibers j of the fifth subset (2.5),
d) defining an error function ƒ to describe the overall deviation between a resulting phase φᵣₑₛ=(φᵢₛ+φₛₑₜ) mod (2π) and the desired modulated phase φ_{des} over all wavelengths λₖ and/or λ_{ƒ} for each of the selected optical fibers j of the fifth subset (2.5),
e) determining the value x_{j_ƒ min} of the correcting variable xⱼ for which the error function ƒ assumes a minimum value for each of the selected optical fibers j of the fifth subset (2.5),
f) providing and positioning an element for compensating for phase distortion of at least two wavelengths λₖ of an image waveguide (1) and/or for implementing at least one optical function which changes propagation directions of electromagnetic radiation of at least one wavelength λ_{ƒ} when entering and/or exiting the image waveguide (1), behind the first end and/or behind the second end of the image waveguide (1), such that the element along the optical axis of each of the selected optical fibers j of the fifth subset (2.5) has the value x_{j_ƒ min} of the correcting variable xⱼ,
and/or
shortening and/or lengthening each selected optical fiber j of the fifth subset (2.5) to compensate for the phase distortion and/or to implement a function which changes propagation directions of electromagnetic radiation when entering and/or exiting the image waveguide (1), at the first end and/or at the second end of the image waveguide (1), such that the shortening and/or the lengthening for each of the selected optical fibers j of the fifth subset (2.5) and each of the wavelengths λₖ and/or λ_{ƒ} has the value x_{j_ƒ min} of the correcting variable xⱼ,
so that the image waveguide (1) comprising the element and/or the shortening and/or lengthening of each of the selected optical fibers j of the fifth subset (2.5) for each of the wavelengths λₖ and/or λ_{ƒ} and each of the selected optical fibers j of the fifth subset (2.5) has a resulting phase φ_{res_ƒ min} in which the error function ƒ assumes a minimal value.
